(19) United States
(12) Patent Application Publication
(10) Pub. No.: US 2019/0336321 A1
(43) Pub. Date: Nov. 7, 2019

(54) INTRAORAL DEVICE

(71) Applicant: Randal P. Garvey, Uhrichsville, OH (US)

(72) Inventor: Randal P. Garvey, Uhrichsville, OH (US)

(21) Appl. No.: 15/968,367

(22) Filed: May 1, 2018

(51) Int. Cl. A61F 5/56; A63B 71/08

(52) U.S. Cl. CPC A61F 5/566; A61F 2005/563; A63B 71/085

(57) ABSTRACT

An intraoral device and method of use for treating conditions of Temporomandibular joint dysfunction (TMD) and Occlusal Muscle Disorder (OMD). A shell is provided including a channel formed with an opening towards a top surface, the channel containing dental impression material enabled to accept a plurality of maxillary anterior teeth of a user. Placing the channel of the intraoral device over maxillary anterior teeth of a user enabling the dental impression material to surround said teeth, cooling the dental impression material forming a mold. A smooth, rigid plane formed on the bottom surface enabled to make contact with mandibular anterior teeth of the user, wherein with the intraoral device in place on the maxillary anterior teeth, the mandibular anterior teeth make contact with the smooth rigid plane and simultaneously translate freely on the smooth rigid plane.

### BACKGROUND OF THE INVENTION

### 1. Field of the Invention

The present invention is in the field of dentistry as it relates generally to an affordable over-the-counter intraoral device for the purpose of providing relief from the pain of Temporomandibular Disorder or Occlusal Muscle Disorder.

### 2. Discussion of the State of the Art

Temporomandibular disorder (TMD) includes conditions of occlusal muscle disorder and intracapsular disorder. These are common terms referring to the pain and dysfunction that results. TMD is a group of symptoms that can occur together which can be caused by anatomical factors, be precipitated by trauma, occlusal changes, or by abnormal or disordered functions referred to as parafunction such as bruxism and clenching.

The mandible, or lower jaw, is articulated with the temporal bone at the temporomandibular joint (TMJ). The TMJ is a complex joint that allows for hinging or rotational action and for gliding or translational movement to open and close. Muscles and teeth related to the TMJ must interact in coordinated harmony to prevent or decrease muscle hyperactivity. If these elements do not function in harmony, pain and restricted movement may occur.

Common symptoms of TMD include pain, clenching, grinding of teeth caused by muscle hyperactivity which may occur when the person is asleep or awake. Most experts strongly recommend treatments that are conservative. Some of these approaches include changing diet, exercise and applying heat to painful areas. Other treatments may include medications such as anti-inflammatory drugs, muscle relaxants, antianxiety and antidepressant drugs; bite splints, mouth guards, or other appliances. Some professionals equilibrate the occlusion in order to remove an occlusal interference.

Muscles of the jaw can be defined as jaw elevators: the masseter, temporalis, medial pterygoid, and superior belly of the lateral pterygoid. Jaw depressors are identified as the anterior digastrics, geniohyoid, mylohyoid and inferior belly of the lateral pterygoid. During jaw opening depressor muscles contract while elevator muscles release. During jaw closing the elevator muscles contract while the depressor muscles release contraction. Specifically, the inferior lateral pterygoid muscle contracts during jaw opening and releases during jaw closing.

Maximum intercuspation refers to the position of the mandible in which the cusps of the teeth of both arches fully interpose themselves with the cusps of the teeth of the opposing arch. When clenching with the condition of maximum intercuspation in centric relation and no posterior interferences, the inferior lateral pterygoid remains in the released or relaxed position.

The inventor of the present invention identifies the cause of TMD as a disruption in the coordinated manner the muscles contract and release, commonly caused by a posterior occlusal interference. Posterior occlusal interferences can be caused by many factors including deflective tooth contact, a condition of tooth contacts that divert the mandible from a normal path of closure. Additionally, a high crown, filling, or wearing of a mouth guard or other oral dental device may cause occlusal interference. Occlusal interference causes discoordination of and directly stimulates hyperactivity of the masticatory neuromusculature, discussed above. Occluso-muscle pain and headache often results rendering the condition of TMD. The specific cause of this pain is the fact that the mandible is not freely moving naturally and the masticatory neuromusculature must compensate for the occlusal interference.

When the mandible is not moving freely and occlusal interference is occurring, the automatic reaction is to move the mandible forward and the muscles contract causing the condyle of the mandible to move out of its natural position, which is seated in the fossa. This muscle contraction is an involuntary reaction to the interference and also happens when one is asleep. The automatic reaction is instigated in order to maintain the natural position, or remove the interference via grinding allowing the mandible to remain in the natural position.

Various non-surgical devices are specifically designed to protect the teeth from damage resulting from TMD and OMD such as “splints”, “mouth guards”, or “night guards”. Over the counter (OTC) mouth guards known in the art are soft full arch coverage devices that will not allow freedom of movement of the mandible. They allow, or actually become posterior interference contacts that limit the mandibular position and cause muscle incoordination, thereby limiting the relief that may be realized by these devices. Full coverage devices hold the mandible in a more forward position, not allowing the condyle to be retracted back into the fossa. This is an inappropriate, incorrect and ineffective way to relieve occlusal muscle pain caused by TMD. These devices actually keep the mandible from moving freely and exacerbate the condition and symptoms of TMD and OCD.

What is needed is an OTC anterior intraoral device has a hard flat, smooth rigid plane on its underside shaped such that only the lower anterior teeth come in contact with the device. This automatically allows freedom of mandibular movement, no posterior tooth contact, proper physiologic position of the condyle, and muscles of mastication to relax, thus eliminating or alleviating muscle pain and tooth wear.

### BRIEF SUMMARY OF THE INVENTION

The present invention provides a new novel intraoral device comprising one embodiment having a shell including a top surface, bottom surface, an anterior edge, a posterior edge and a depth between the top and bottom surfaces. A channel formed within the depth along the anterior edge, opening towards the top surface, the channel containing dental impression material enabled to accept a plurality of maxillary anterior teeth of a user.

A smooth, rigid plane may be formed on the bottom surface of the intraoral device enabled to make contact with mandibular anterior teeth of the user. When the intraoral device is in place on the maxillary anterior teeth, the mandibular anterior teeth make contact with the smooth rigid plane, when the jaw is in a closed position, and simultaneously translates freely on the smooth rigid plane.

Another embodiment provides that, with the device in place on the maxillary anterior teeth, a space is formed between maxillary and mandibular molars of the user preventing any contact between the molars. An additional embodiment includes heating the dental impression material prior to inserting the maxillary anterior teeth, cooling the material, removing the maxillary anterior teeth thereby forming a mold of the maxillary anterior teeth in the channel for future use.

The maxillary anterior teeth mentioned above, include right and left central incisors, right and left lateral incisors and right and left canine teeth and the mandibular anterior teeth include at least central incisors, lateral incisors and cuspids or canines. The channel may also include a posterior wall with a first height and an anterior wall with a second height and the first height is less than the second height.

In an alternative embodiment, the smooth rigid plane, when in position on the maxillary anterior teeth is positioned to be parallel with a bite plane defined by all of the maxillary teeth. An additional option includes a tether opening enabled to secure a tether surrounding the users head while the device is in place on the maxillary anterior teeth.

The tether may also be manufactured from medical grade silicone cord. In one embodiment the tether extends from a centered position on the smooth rigid plane at the anterior edge of the shell.

A method of treating conditions of Temporomandibular disorder (TMD) with an intraoral device is also provided comprising the steps of (a) providing a shell including a top surface, bottom surface, an anterior edge, a posterior edge and a depth between the top and bottom surfaces; (b) heating dental impression material held within a channel formed within the depth along the anterior edge, opening towards the top surface; (c) placing the channel of the intraoral device over maxillary anterior teeth of a user enabling the dental impression material to surround said teeth, cooling the dental impression material forming a mold; and (d) maintaining a space between maxillary and mandibular molars while the intraoral device is in place on the maxillary anterior teeth via mandibular anterior teeth making contact with a smooth rigid plane formed on the bottom surface of the intraoral device thereby allowing the mandibular anterior teeth to make contact with the smooth rigid plane while simultaneously freely translating along the plane.

One embodiment of the method includes that the intraoral device is held in place while cooling enabling the smooth rigid plane to be parallel with a bite plane defined by all of the maxillary teeth. Additionally, the maxillary anterior teeth may include left and right central incisors, left and right lateral incisors and left and right canine teeth. The device may include more or less of the maxillary anterior teeth. The mandibular anterior teeth may include at least central incisors, lateral incisors and cuspids or canines.

An alternative embodiment may include a posterior and anterior wall forming the channel, wherein the posterior wall has a first height and the anterior wall has a second height and the first height is less than the second height. The intraoral device may also include a tether opening enabled to secure a tether surrounding the users head while the device is in place on the maxillary anterior teeth. The tether may be manufactured from medical grade silicone cord and extend from a centered position on the smooth rigid plane at the anterior edge of the shell.

### BRIEF DESCRIPTION OF THE SEVERAL VIEWS OF THE DRAWINGS

FIG. 1A is a perspective view of the intraoral bite plane device showing the device after the dental impression material has been customized by the user.

FIG. 1B is a plan view of the intraoral bite plane device customized by the user.

FIG. 2A provides a bottom view of the maxillary teeth and palate.

FIG. 2B is a left side view of the jaw, and identifies the left mandibular teeth.

FIG. 3A is a left side view of the skull identifying bone structure of the jaw area.

FIG. 3B provides an enlarged left side view of the temporomandibular joint in a closed or relaxed position.

FIG. 3C provides an enlarged left side view of the temporomandibular joint in an open position with the condyle and articular disc displaced from the glenoid fossa.

FIG. 4 shows a left side view of the skull and the four primary muscles of mastication.

FIG. 5A is a front view of the shell of the device.

FIG. 5B is a plan or top view of the shell of the device.

FIG. 6A is a posterior top view of the shell of the device.

FIG. 6B is a posterior bottom view of the shell of the device.

FIG. 7A is a left side view of the shell of the device.

FIG. 7B is a left side section view of the shell of the device.

FIG. 8A provides a bottom view of the device in place on the anterior maxillary teeth.

FIG. 8B shows a left side view of the device prepared and aligned to form the teeth impressions.

FIG. 9A shows a left side view of the device in place over the anterior maxillary teeth during forming and when in use.

FIG. 9B shows a left side view of the device in place over the anterior maxillary teeth with the anterior mandibular teeth resting against the flat underside of the device.

### DETAILED DESCRIPTION OF THE INVENTION

The present inventor identifies and addresses a problem in the art not considered by current art professionals when developing devices that only address the symptoms of TMD. As previously presented, the actual root cause of TMD and OMD is a disturbance in the coordinated muscle function of masticatory musculature controlling the mandible, condyle and articular disc. Only when the mandible is left to translate freely during treatment can the root cause of TMD and OMD be corrected.

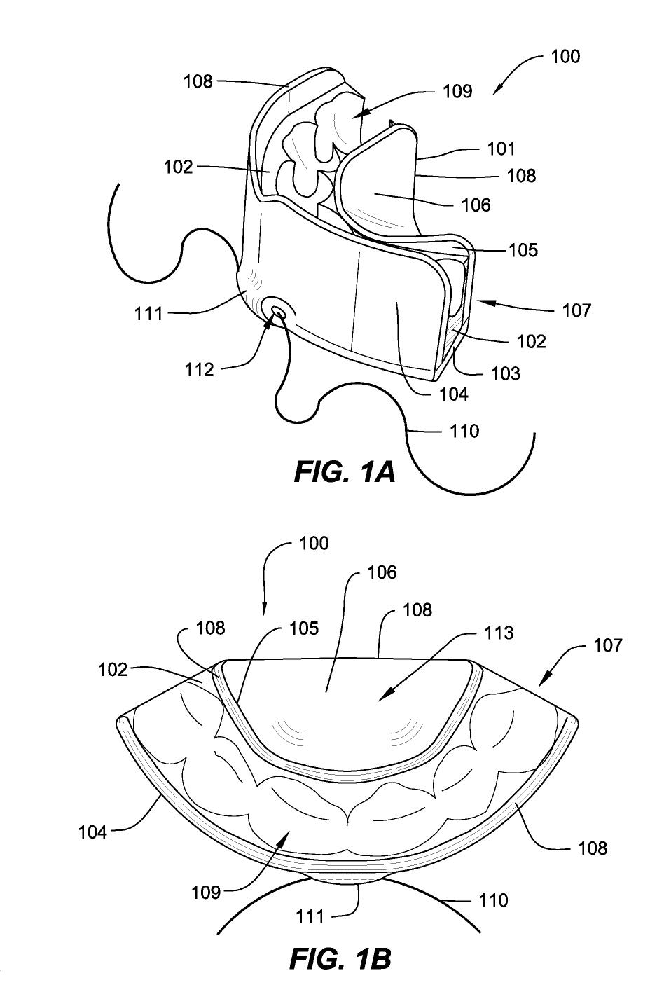

FIGS. 1A and 1B depict a preferred embodiment of an OTC intraoral device 100 providing relief from acute discomfort of temporomandibular dysfunction (TMD) at a lower cost, without waiting for a dental appointment. Device 100 is comprised of an outer shell 101, dental impression material 102, and a smooth flat rigid plane 103. Shell 101 is comprised of curved outer channel wall 104 which is slightly larger than and parallel to the labial or outside surface of the upper teeth; curved inner channel wall 105 slightly smaller than but parallel to the lingual or inside surface of the upper teeth; and a curved transition surface 106 situated within wall 105 sloping down to the posterior edge of plane 103.

Walls 104 and 105, may be joined with plane 103 during fabrication of device 100 by molding, by adhesion, by over molding, or other manufacturing processes. These elements form channel 107 into which a predetermined amount of material 102 is placed by the manufacturer. When impression material 102 is heated to become formable, for example, in hot water, impressions 109 of the teeth are made in the material, as described later. This material may be heated and reheated as necessary, or may be heated and formed one time.

All exposed edges 108 of device 100 shall be formed to a smooth radius to prevent irritation to the user's cheeks, gums, and tongue. Device 100 is equipped with an adjustable tether 110 that surrounds the head while device 100 is in use so that it can be retrieved if dislodged and swallowed or aspirated. Tether 110 shall be manufactured of medical grade silicone cord or similar flexible material. Tensile strength of tether 110 could be low enough that there is no danger of strangulation. A tether attachment tab 111 with cylindrical hole 112 allows the tether to be secured to device 100. The tether may be replaced if it is broken. Tether 110 is not shown in remaining figures to reduce confusion. Space 113 may provide a comfortable space for the user's tongue.

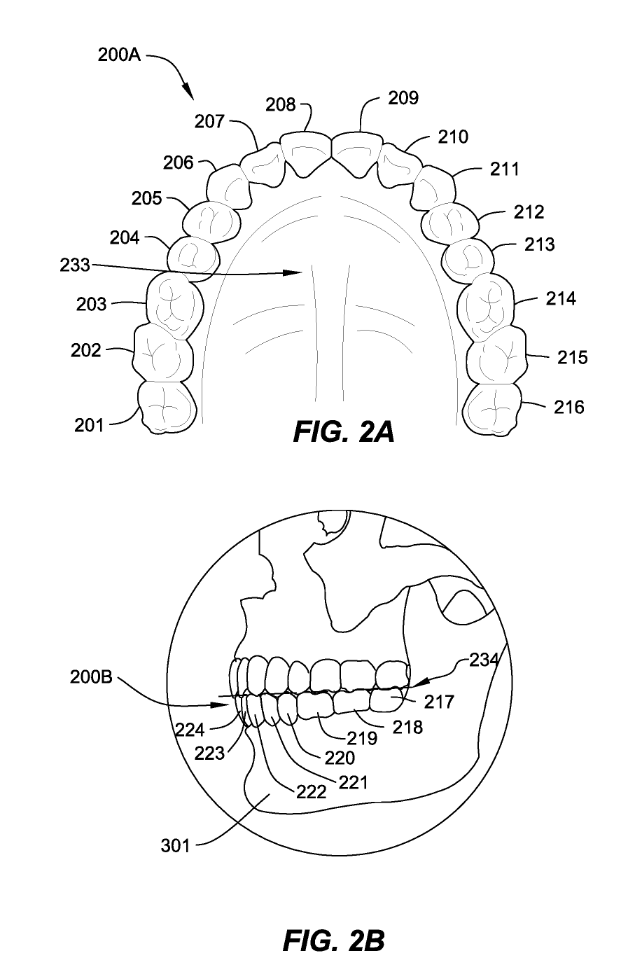

FIG. 2A identifies the maxillary or upper teeth 200A. Teeth 200A starting at the anterior center include: Central incisors 208 and 209, lateral incisors 207 and 210, cuspids, canine or eye teeth 206 and 211, first premolars or first bicuspids 205 and 212, second premolars and second bicuspids 204 and 213, first molars 203 and 214, second molars 202 and 215, and third molars 201 and 216 or wisdom teeth. The hard palate 233 or “roof” of the mouth is indicated as shown.

Likewise FIG. 2B identifies in a side view of mandibular or lower teeth 200B, left side only; starting at the front are the left central incisor 224, lateral incisor 223, cuspid or canine 222, first premolar 221, second premolar 220, first molar 219, second molar 218, and third molar 217. Corresponding mandibular teeth on the right side of the mouth are not shown in this view. The mandible or lower jaw is shown at 301. The occlusal plane 234 indicated in this figure is the plane that is formed by the occlusal surfaces of the teeth. Occlusal plane 234 is important when considering the function of the TMJ and in planning treatments to optimize the outcome for patients. This occlusal plane will be referred to again in later figures.

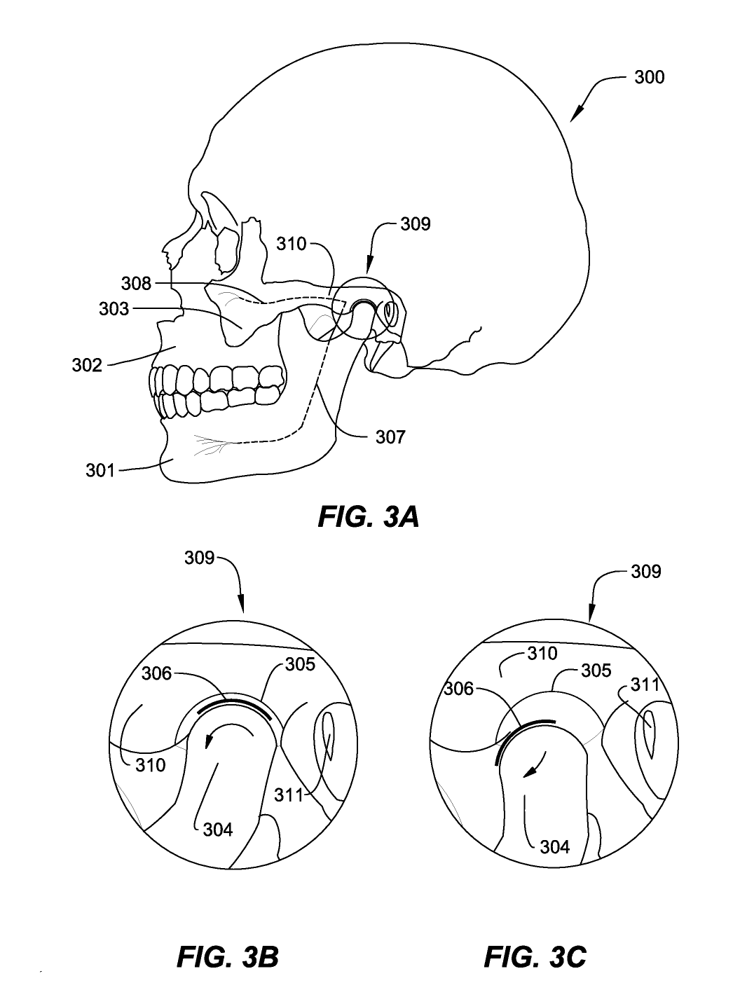

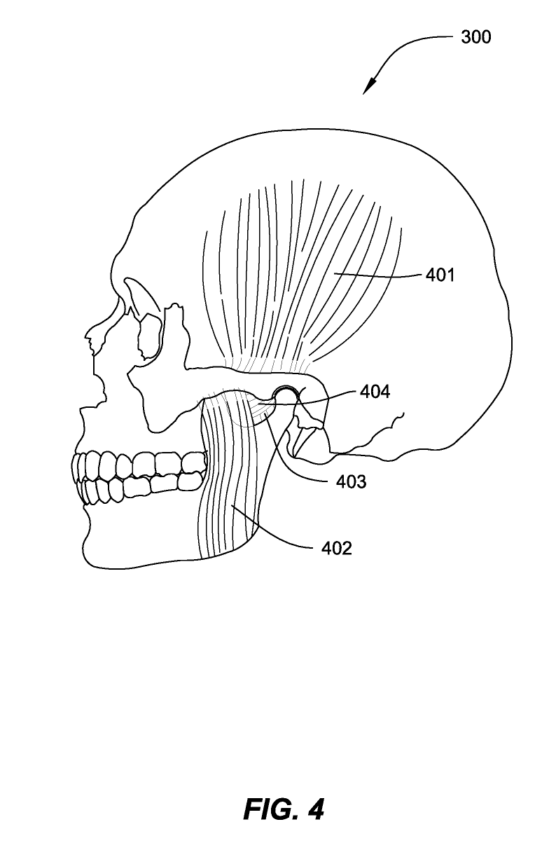

FIG. 3A shows a left side view of the human skull 300. Identified thereon are the mandible or lower jaw 301, the maxilla 302, and the zygomatic bone 303. The right side of the skull has the same corresponding features which are not shown. Mandible 301 has a curved arch form, with wide ramus portions which extend from the angles of the mandible upward toward the temporomandibular joint on each side in front of ear openings 311 (FIG. 3B). Mandible 301 moves while talking, eating, clenching or grinding. Mandible 301 supports lower teeth 200B and shapes the chin and jowl areas of the face. Maxilla 302 supports upper teeth 200A. Zygomatic 303 shapes the cheek. Mandibular nerve 307 and maxillary nerve 308 run along the inside of mandible 301 and maxilla 302 until they surface outside of 301 and 302 and branch off into numerous smaller nerves as indicated. Nerve 307 innervates the muscles of mastication which are identified later in FIG. 4. TMJ 309 is indicated in this view and further detail is shown in the following figures.

FIG. 3B provides an enlarged left side view of joint 309 when it is in a relaxed state, such as when the jaw is closed and in a fully seated position where the condyle sits wholly in the fossa. Mandible 301 (FIG. 3A) articulates with the temporal bone in front of ear opening 311. Condyle 304 tapers in width and ends in a rounded head as shown. Temporal bone 310 is joined to zygomatic 303 by an oblique suture. Bone 310 continues along the high side of the check over opening 311. In front of opening 311, bone 310 has a concave depression that is the glenoid fossa 305. In the upper most position (centric relation) condyle 304 is positioned and braced against the temporal bone under the disc inside fossa 305. Articular disc 306 is a thin disc of nonvascular fibrous connective tissue that provides a cushion between condyle 304 and fossa 305. As mandible 301 opens slightly, it rotates about condyle 304 as indicated in this figure by the rotational arrow. As the opening increases condyle 304 begins its forward translation.

FIG. 3C provides an enlarged left side view of joint 309 when it is in an open normal physiologic state. This view shows that mandible 301 translates down and forward on the disc as indicated by the arrow in this figure. Condyle 304 and disc 306 slide out of fossa 305 as shown. Joint 309 has a fibrous capsule surrounding the joint and a number of ligaments associated with the structure of the joint which are not shown in these figures. Stretching of those ligaments may cause corresponding symptoms such as popping, clicking, grating noises, tinnitus, and ear pain. The posterior of disc 306 is attached to fossa 305 with retrodiscal tissue not shown in the figures. The retrodiscal tissue is vascular and highly innervated. It can be painful if it is inflamed or compressed when the disc is displaced in a forward position in the joint. If inflammation lasts long enough adhesions and scar tissue may form causing more pain.

FIG. 4 shows skull 300 and depicts the four primary muscles of mastication which are temporalis muscle 401, masseter muscle 402, medial pterygoid muscle 403, and lateral pterygoid muscle 404. Muscles 403 and 404 are both situated on the inside surface of mandible 301 so only a small portion can be seen in this figure. Muscle 402, together with muscle 403, form a sling around mandible 301. Muscles 401, 402 and 403 are used to adduct or raise the jaw. Muscle 404 protrudes the mandible and the disc in a coordinated movement. As indicated in the previous discussion of FIG. 3A, nerve 307 innervates these muscles of mastication.

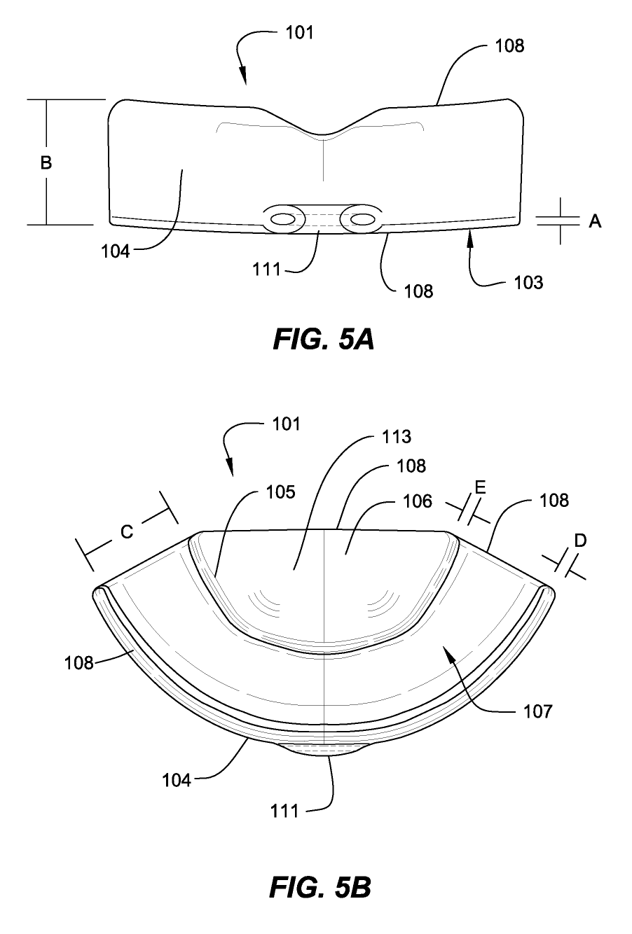

FIG. 5A provides a front view of shell 101 without material 109. Rigid plane 103 forms the bottom of device 100 and may be constructed of a medical grade polymer material with rigidity such that the plane will not flex under pressure when in use. The surface of plane 103 should be smooth to the point of providing little or no resistance to upper edges of the bottom anterior teeth that make contact with it, thus allowing the upper edges of the teeth to translate freely. There are a variety of materials that may be used to manufacture the plane 103 including, but not limited to enamel, acrylic, and synthetic polymers and resins with a smooth glass-like surface and durable enough to withstand continued contact from enameled teeth. Some metals are smooth enough to allow the lower anterior teeth to make contact and slide freely as well, including polished titanium and stainless steel.

Dimension A is the thickness of plane 103 which should be minimized to avoid adding bulk to device 100. Dimension A could range from 0.5 mm to 1.5 mm depending on specific type of material used. Materials comprising walls 104 and 105 (not shown in this view) and the mass forming surface 106 may be constructed of medical grade polymer however this material may be slightly malleable to make device 100 more comfortable for the user. Dimension B is the height of wall 104. This height may be in the range of 6 mm to 8 mm; it will not rise above the labial gingival margin of teeth 200 to minimize user discomfort or tissue irritation.

FIG. 5B shows a plan or top view of shell 101. The outline of shell 101 is the same shape as plane 103 which forms the underside of device 100. Plane 103 cannot be seen in this view, but is shown in FIG. 5A above. As can be seen in FIG. 5B, walls 104 and 105 are arranged along arcs which parallel just inside and outside of upper teeth 200 (FIG. 2A) respectively. Dimension C is the width of channel 107 formed by walls 104 and 105 and plane 103 which encompasses the six anterior maxillary teeth 206-211. FIG. 1B outlines these teeth as impression 109. Walls 104 and 105 extend to the interproximal or interdental interface between teeth 205 and 206 and between teeth 211 and 212. Tab 111 can be seen in this view. The tab is rounded and smooth in all directions to prevent irritating contact with the user's lips. Dimension D is the thickness of wall 104 which may be in the range of 1 mm-3 mm, but may be the minimum required to retain the shape of channel 107 during the forming process described later herein. Wall 105 is supported by the mass of material which forms surface 106. Dimension E depicts the thickness of wall 105 which may be formed with a radius and thickness as dimension D.

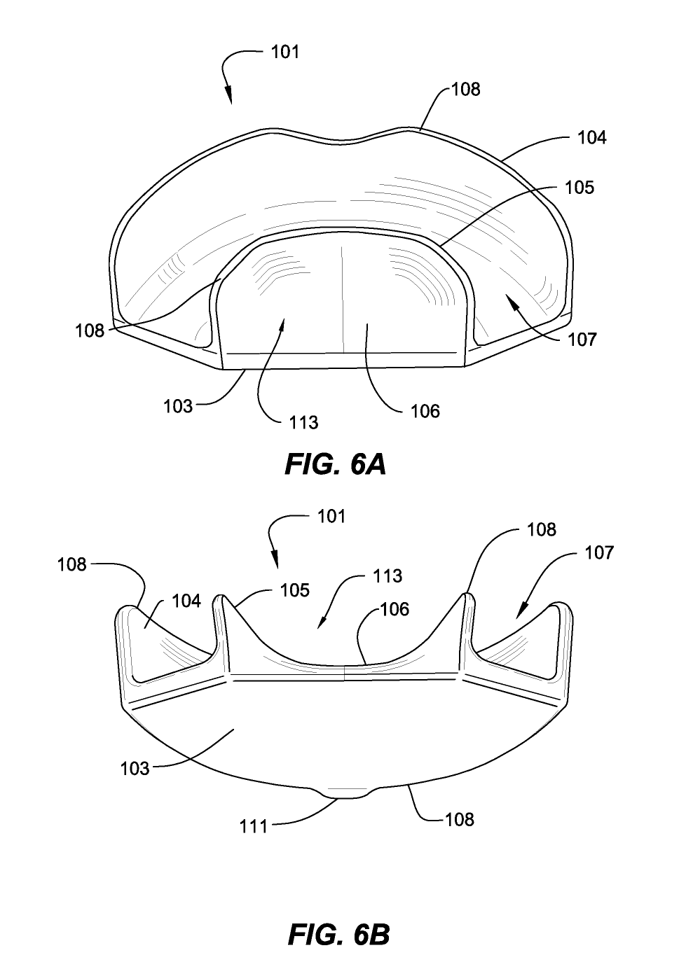

FIGS. 6A and 6B show shell 101 from posterior above and below to provide clearer understanding of the geometry of device 100. Space 113 is more clearly shown in this view. The user may extend the tongue if needed in space 113 above device 100 between surface 106 and palate 233, or below plane 103 in the space surrounded by lower teeth 200B.

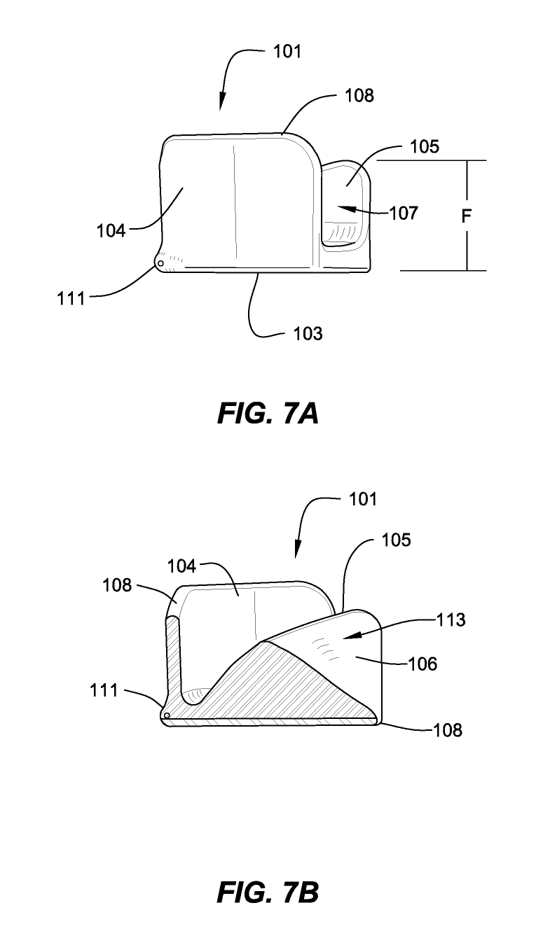

FIG. 7A provides a left side view of shell 101. Dimension F is the height of wall 105. As can be seen, dimension F is less than dimension B on FIG. 5A, and may be in a range of 4 mm to 7 mm. Wall 105 is lower than wall 104 because the gingival margin on the lingual side of teeth 206-211 is lower than on the labial side.

FIG. 7B is a left side section view of shell 101 cut along the front to back centerline. This figure provides additional clarification of the contour of surface 106 and space 113. In this view the device is placed over teeth 206-211. More or less teeth may be incorporated in the device while still enabling regular function of the teeth as understood by one with skill in the art. For example, device 100 may only incorporate teeth 207-210 or 205-212.

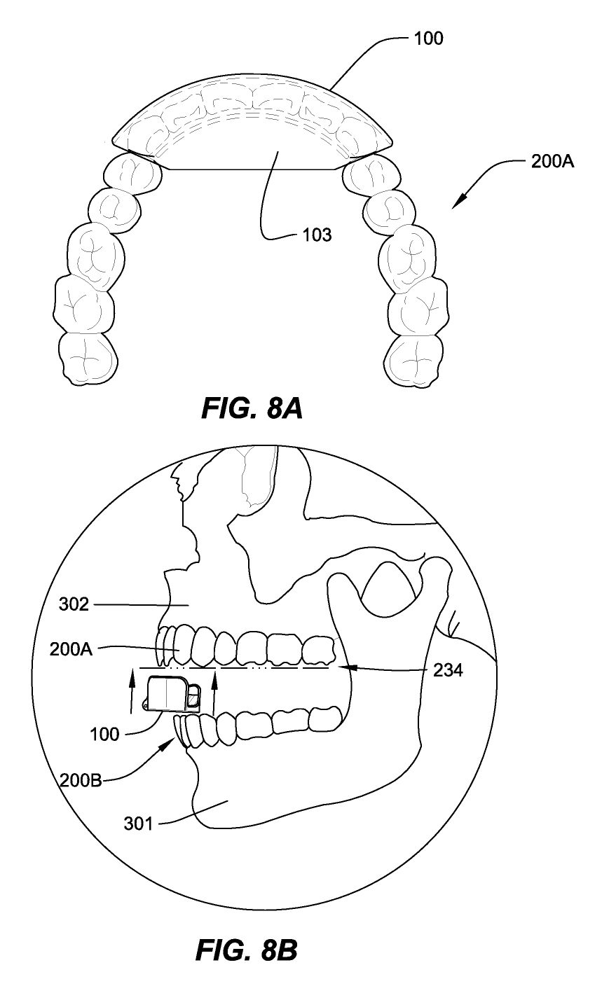

FIG. 8A provides a bottom view of maxilla 202 and teeth 200A with device 100 properly in place. Surface 103 is toward the viewer. As shown in this embodiment, the device covers teeth 206-211 (FIG. 2A).

FIG. 8B shows a left side view wherein device 100 is ready to fit to the user. Shell 101 is shown with a premeasured volume of material 102 in channel 107. The amount of material is selected to fill the channel 107 more than half the depth of the channel in order to cover the teeth when formed to an extent that holds the device 100 on the teeth securely.

The user may heat device 100 in a microwave or in hot or boiling water to soften material 102, depending upon the type of material used. After a recommended period of time to cool device 100 and material 102 to a safe temperature, device 100 is inserted into the mouth placing it over teeth 206-211, in this embodiment, and pressing device 100 firmly in place in the direction of the arrows shown. Surface 103 may be oriented to align as closely as possible with plane 234 for proper fit. The user may use thumbs to hold device 100 in place while material 102 continues to cool and become firmer based on manufacturer recommendations. Material 102 shall be of medical grade thermoplastic with a low enough melting point that it can formed at a low enough temperature that it is safe to handle and to use to create the impressions. Thermoplastic material may be used that can be reheated and reformed in the event the user does not initially achieve the desirable fit the first time.

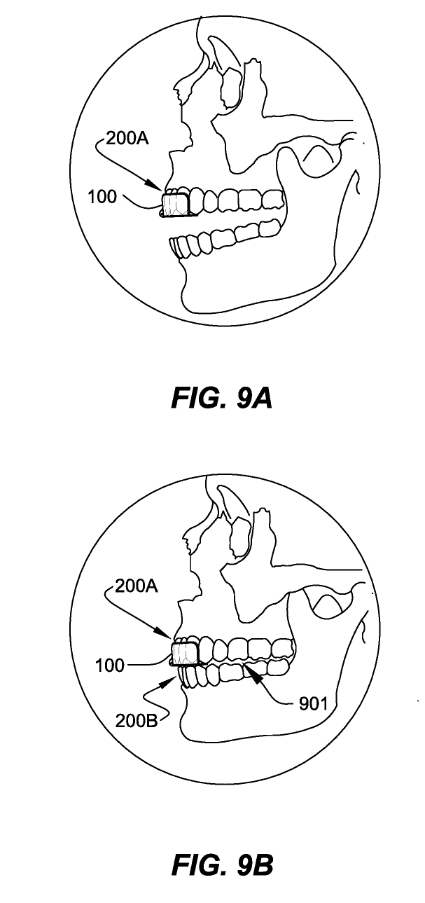

FIG. 9A is a left side view showing device 100 properly situated over anterior teeth 206-211 in this embodiment. Displacement of material 102 by teeth 206-211 create impressions 109 which snugly fit around teeth securing device 100 in place during use. The premeasured volume of material 102 will be such that it is not pressed against the gums to avoid discomfort but will sufficiently encompass and provide secure fit around the affected teeth.

FIG. 9B is a left side view showing device 100 in place with any or all of the anterior mandibular teeth making contact with plane 103 of device 100. The thickness of plane 103 together with the irregularities of the occlusions of lower teeth 222-225 in contact with the plane hold the mandible and maxilla slightly apart, creating an interocclusal space 901 between the maxillary and mandibular premolars and molars. In this position, muscles 401-404 are able to relax and condyle 304 and disc 306 return to a natural position in relation to fossa 305. Additionally the mandible retains its freedom of movement. Only with an OTC device as provided by the present inventor, can the muscles of mastication relax into coordinated function, while maintaining complete freedom of movement of the mandible and all components included in 309.

The benefits realized by the present invention includes stopping uncoordinated muscle hyperactivity by parting the back teeth thus eliminating premature interferences of posterior (back) teeth in centric relation. The flat, smooth, rigid plane allows freedom of movement of the mandible (permissive). Therefore, when the muscle incoordination is eliminated, the mandible is free to move and be seated correctly in the fossa. When the muscle hyperactivity and incoordination is eliminated, the muscle pain subsides. The muscles relax and muscle splinting subsides. When the muscle incoordination and hyperactivity is eliminated, the disc is allowed to be pulled back (posteriorly) into a more normal position onto the head of condyle, or at least help keep the disc from being pulled more forward in the early stages of TMD. Device 101 can stop or slow down the progression of TMJ internal derangements. Because the posterior teeth are held apart, they are protected from injury (fracture), wear, and bone loss from heavy occlusal forces from systemic and nocturnal bruxing or clenching caused by the hyperactiviy.

It will be apparent to one with skill in the art that there may be variant architectures and hardware additions that may be provided to the TMJ bite plane without departing from the spirit and scope of the present invention. The device may also be manufactured in at least two or more sizes to provide better fit to a greater number of users. Another embodiment might be produced for use by professionally licensed dentists.

It will be apparent to the skilled person that the arrangement of elements and functionality for the invention is described in a preferred embodiment which is exemplary of an implementation of the invention. These exemplary descriptions do not preclude other implementations and use cases not described in detail. The elements and functions may vary, as there are a variety of ways the device may be implemented within the scope of the invention. The invention is limited only by the breadth of the claims below.

1. An intraoral device, comprising;
a shell including a top surface, bottom surface, an anterior edge, a posterior edge and a depth between the top and bottom surfaces;
a channel formed within the depth along the anterior edge, opening towards the top surface, the channel containing dental impression material enabled to accept a plurality of maxillary anterior teeth of a user; and
a smooth, rigid plane formed on the bottom surface enabled to make contact with mandibular anterior teeth of the user;
wherein with the intraoral device in place on the maxillary anterior teeth, the mandibular anterior teeth make contact with the smooth rigid plane and simultaneously translate freely on the smooth rigid plane.

2. The intraoral device of claim 1, wherein with the device in place on the maxillary anterior teeth, a space is formed between maxillary and mandibular molars of the user preventing any contact between the molars.

3. The intraoral device of claim 1, wherein the dental impression material is heated prior to inserting the maxillary anterior teeth, cooling the material, removing the maxillary anterior teeth thereby forming a mold of the maxillary anterior teeth in the channel for future use.

4. The intraoral device of claim 1, wherein the maxillary anterior teeth include right and left central incisors, right and left lateral incisors and right and left canine teeth.

5. The intraoral device of claim 1, wherein the mandibular anterior teeth include at least central incisors, lateral incisors and cuspids or canines.

6. The intraoral device of claim 1, wherein the channel includes a posterior wall with a first height and an anterior wall with a second height and the first height is less than the second height.

7. The intraoral device of claim 1, wherein the smooth rigid plane, when in position on the maxillary anterior teeth is positioned to be parallel with a bite plane defined by all of the maxillary teeth.

8. The intraoral device of claim 1, including a tether opening enabled to secure a tether surrounding the users head while the device is in place on the maxillary anterior teeth.

9. The intraoral device of claim 4, wherein the tether is manufactured from medical grade silicone cord.

10. The intraoral device of claim 5 wherein the tether extends from a centered position on the smooth rigid plane at the anterior edge of the shell.

11. A method for treating conditions of Temporomandibular disorder (TMD) and Occlusal Muscle Disorder (OMD) with an intraoral device comprising the steps of:
(a) providing a shell including a top surface, bottom surface, an anterior edge, a posterior edge and a depth between the top and bottom surfaces;
(b) heating dental impression material held within a channel formed within the depth along the anterior edge, opening towards the top surface;
(c) placing the channel of the intraoral device over maxillary anterior teeth of a user enabling the dental impression material to surround said teeth, cooling the dental impression material forming a mold;
(d) maintaining a space between maxillary and mandibular molars while the intraoral device is in place on the maxillary anterior teeth via mandibular anterior teeth making contact with a smooth rigid plane formed on the bottom surface of the intraoral device thereby allowing the mandibular anterior teeth to make contact with the smooth rigid plane while simultaneously freely translating along the plane.

12. The method of claim 11, wherein in step (c) the intraoral device is held in place while cooling enabling the smooth rigid plane to be parallel with a bite plane defined by all of the maxillary teeth.

13. The method of claim 11, wherein the maxillary anterior teeth include left and right central incisors, left and right lateral incisors and left and right canine teeth.

14. The method of claim 11, wherein the mandibular anterior teeth include at least central incisors, lateral incisors and cuspids or canines.

15. The method of claim 11, wherein the channel includes a posterior wall with a first height and an anterior wall with a second height and the first height is less than the second height.

16. The method of claim 11, including a tether opening enabled to secure a tether surrounding the users head while the device is in place on the maxillary anterior teeth.

17. The method of claim 17, wherein the tether is manufactured from medical grade silicone cord.

18. The method of claim 18 wherein the tether extends from a centered position on smooth rigid plane at the anterior edge of the shell.
